(19) United States
(12) Patent Application Publication
(10) Pub. No.: US 2006/0051291 A1
(43) Pub. Date: Mar. 9, 2006

(54) Synthesis of radiolabeled sugar metal complexes

(76) Inventors: Michael J. Adam, Surrey (CA); Cara L. Fisher, Surrey (CA); Simon R. Bayly, Islip (GB); Christopher Orvig, Vancouver (CA); Nathaniel C. Lim, Vancouver (CA); Timothy J. Storr, Vancouver (CA); Charles B. Ewart, Vancouver (CA)

Correspondence Address: HARNESS, DICKEY & PIERCE, P.L.C., P.O. BOX 8910, RESTON, VA, 20195, US

(21) Appl. No.: 11/219,846

(22) Filed: Sep. 7, 2005

Related U.S. Application Data
(60) Provisional application No. 60/607,295, filed on Sep. 7, 2004.

(51) Int. Cl. A61K 51/00; C07F 13/00

(52) U.S. Cl. 424/1.11; 534/14

(57) ABSTRACT

The invention provides a method for manufacturing or preparing neutral, low molecular weight ^{99m}Tc-labeled and ¹⁸⁶Re-labeled carbohydrate complexes with an improved radiochemical yield from a simple functionalized sugar, such as glucosamine. In particular the synthesis relies on single ligand transfer (SLT) or double ligand transfer (DLT) reactions for converting a ferrocene compound into a rhenium or technetium tricarbonyl complex. The ferrocene compound may be linked to a sugar through various functional groups including, for example, thio, amino and alcohol functionalities to provide a wide range of radiolabeled sugar complexes that include both water soluble and relatively water insoluble compounds.

### PRIORITY STATEMENT

This application claims priority pursuant to 35 U.S.C. § 119 from U.S. Provisional Application No. 60/607,295, filed Sep. 7, 2004, the content of which is incorporated, in its entirety, herein by reference.

### BACKGROUND OF THE INVENTION

1. Field of the Invention

The invention relates to methods for producing radiolabeled sugar metal complexes and the resulting radiolabeled materials.

2. Description of Related Art

Radiolabeled carbohydrates have been of increasing interest in nuclear medicine applications due, in part, to the success of 2-¹⁸F-fluoro-2-deoxy-glucose (FDG) as an imaging agent in positron emission tomography (PET). The success of FDG is attributable, in part, to its utility for imaging both cardiac viability and tumors due to the fact that it undergoes glucose metabolism and is a substrate for hexokinase. This success has raised the question of whether a single-photon emitting glucose analog with properties and utility similar to FDG can be developed for use with single-photon emission computed tomography (SPECT). Because of the relatively short half life of ¹⁸F (110 minutes), its use is limited to facilities that have an accelerator in close proximity to chemistry laboratories and medical facilities, thereby rendering the FDG method impractical for wide use in medical applications.

By comparison, ^{99m}Tc, an isotope perhaps most commonly used in SPECT applications, may be produced as Na^{99m}TcO₄ from a ⁹⁹Mo generator making it widely available and relatively inexpensive. The third row transition metal analogue of technetium, rhenium, has similar chemistry to that of technetium and has particle emitting radioisotopes with physical properties applicable to therapeutic nuclear medicine. For these reasons, a ^{99m}Tc SPECT tracer that will mimic the biodistribution of FDG and the therapeutic potential of the analogous rhenium compounds may be particularly useful. Although ^{99m}Tc is widely used in imaging applications, one complication to address in preparing a tracer is that this isotope must be attached to the molecule via a chelate or organometal conjugate, which may perturb the system being studied.

A SPECT analog based on a widely available isotope such as ^{99m}Tc would make these agents available to the broader medical community. Among elements of the same series as Tc the isotopes ^{186/188}Re also show promise in the development of therapeutic strategies. For a β⁻ emitting radioelement to be therapeutically useful, a half-life of between 12 hours and 5 days is preferred. Moreover, for a 1 MeV β⁻ particle, the depth of penetration into tissue is approximately 5 mm. Furthermore, if some of the disintegrations are accompanied by emission of a 100-300 keV gamma photon, the behavior of the radioelement can be conveniently followed by using a gamma camera. The nuclear properties of ^{186/188}Re are well suited for these purposes.

There remains considerable interest in and need for improved radio-metal, carbohydrate derivatives that can be used as imaging agents and/or therapeutic agents in neurology, cardiology and oncology. In particular, the development of techniques for the synthesis of ^{99m}Tc, ^{186/188}Re-labeled sugars via sugar-ferrocenyl or sugar-chelate derivatives are of interest.

There have been several recent reports on the synthesis of ^{99m}Tc-labeled and ^{186/188}Re-labelled organic pharmaceuticals, such as steroids, tropanes, peptides and others, for use in imaging the brain and other organs with SPECT. One of the more successful efforts has produced ^{99m}Tc-TRODAT, a dopamine reuptake inhibitor that is useful in imaging patients with Parkinson's Disease. This compound is a spinoff product of the research on ¹⁸F-labeled and ¹¹C-labeled tropane analogs that have been used as PET imaging agents to study movement disorders. Researchers at several centers have also been working over the years on the development of tropane PET imaging agents to study the dopaminergic system. It was from an extension of this work that a ^{99m}Tc-analog was synthesized that allowed this research to be carried out by a broader medical community using SPECT. Surprisingly, the attachment of the relatively large molecular weight Tc-BAT (bis(aminoethanethiol)) metal complex (C₄H₁₂N₂S₂OTc) to the tropane derivative does not destroy the receptor binding capability of the drug.

### BRIEF DESCRIPTION OF THE INVENTION

The invention provides a method for manufacturing or preparing neutral, low molecular weight ^{99m}Tc-labeled and ¹⁸⁶Re-labeled carbohydrate complexes with an improved radiochemical yield from a simple functionalized glucosamine.

### BRIEF DESCRIPTION OF THE PATENT DRAWING

Analysis of representative products was performed using HPLC with a solvent consisting of 0.1% trifluoroacetic acid in water (solvent A) and acetonitrile (solvent B). Samples were analyzed with a linear gradient method (100% solvent A to 100% solvent B over 30 minutes). The results of this HPLC analysis are reflected below in the Figure.

### DETAILED DESCRIPTION OF EXAMPLE EMBODIMENTS

Rhenium carbonyl complexes of β-estradiol derivatives, in which a chromium-tricarbonyl moiety was either attached to the aromatic ring of the steroid or as a cyclopentadienyl chromium tricarbonyl pendant group to the 17α position, have been shown to have high affinity for the estradiol receptors. The synthesis of a 5-HT_{1A} serotonin brain receptor ligand labeled with ^{99m}Tc has also been achieved with the technetium-tricarbonyl moiety attached via chelation to the neutral bidentate amine ligand (NˆN′) portion of the molecule.

Another use of ^{99m}Tc in medicine involves the labeling of a cyclopentadienyltricarbonyl-[^{99m}Tc]-tropane conjugate using a technique to achieve a double ligand transfer (DLT) (synthesis I) or a single ligand transfer (SLT) (syntheses II and III), as illustrated below, to convert a ferrocene compound into a rhenium- or technetium-tricarbonyl complex. Because the only available chemical form of radioactive Re and Tc is as ReO₄⁻ or TcO₄⁻, many rhenium and technetium 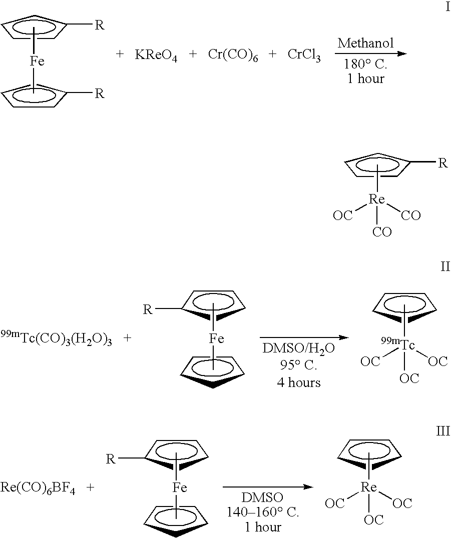
radiopharmaceuticals are inorganic complexes with the metal in the +5 oxidation state. The DLT and SLT reactions opens up the possibility of forming (cyclopentadienyl)tricarbonyl-technetium and -rhenium organometallic radiopharmaceuticals from the perrhenate and pertechnetate forms of these isotopes. Due to the harsh conditions of the DLT reaction, more success has been achieved in synthesizing sugar-Cp complexes with Tc or Re using an indirect approach as shown below (synthesis IV). 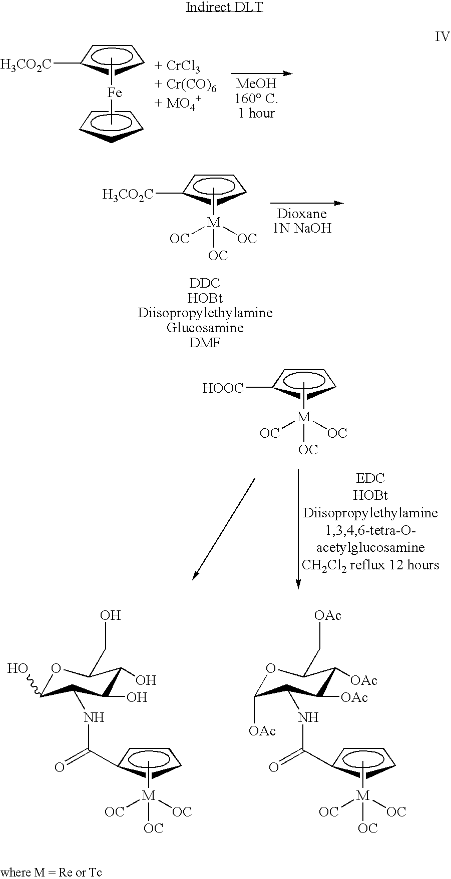
However, by applying the SLT reaction it was possible to synthesize sugar metal Cp derivatives of Tc using the ISOLINK boranocarbonate kit as shown below, in 50-70% radiochemical yield.

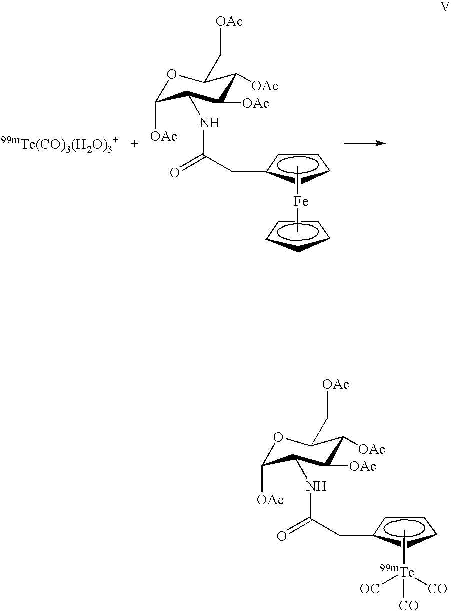

Ferrocene can be synthesized with a wide variety of functionality on one or both of its cyclopentadienyl rings. As a result, ferrocenyl-sugar conjugates, including, for example, the dozen conjugates illustrated below, may be successfully prepared giving the SLT reaction significant potential.

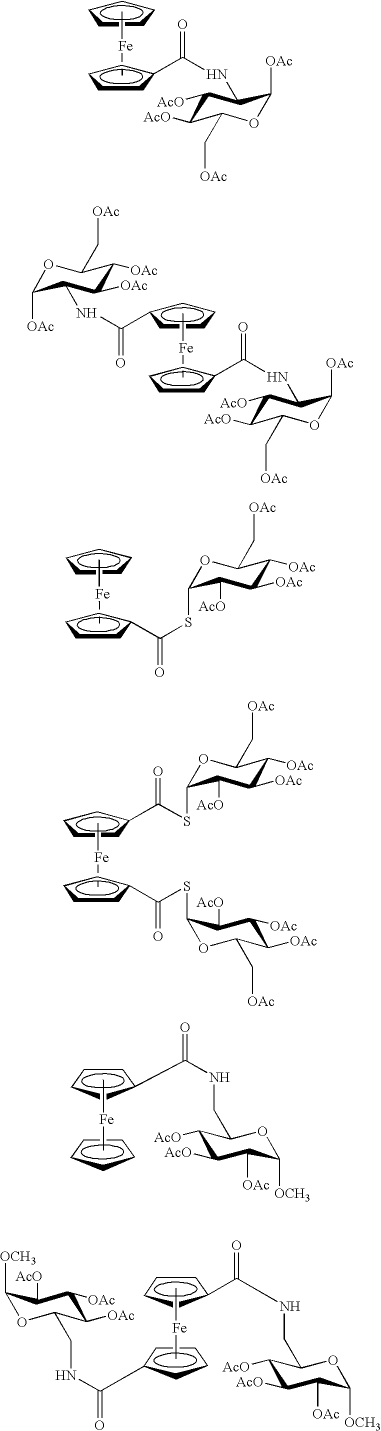

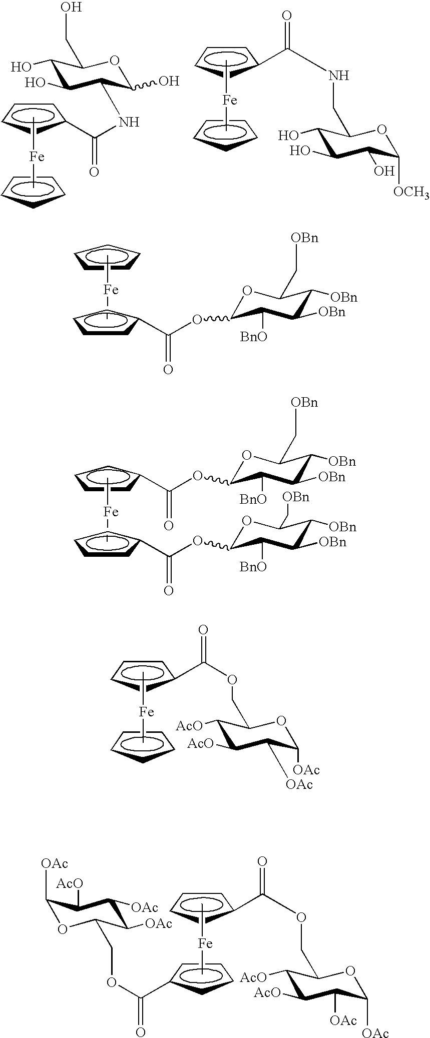

Ferrocene may then be linked to these sugars through thio, amino and/or alcohol functionalities present on the sugars. The sugars were either fully protected, yielding organic soluble ferrocene derivatives, or were unprotected, resulting in water soluble conjugates.

Tc- and Re-Sugars Via Metal Chelates

A number of sugar-metal chelates based on Schiff base complexes have previously been synthesized from glucosamine derivatives with salicylaldehyde or 3-aldehydo-salicylic acid. Using these ligands, it was possible to form a number of complexes using Cu, Zn and Co as the metal. A generic example of such a complex is shown in below with M representing the metal:

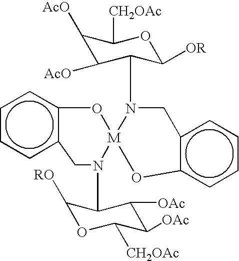

Recent efforts have demonstrated that carbohydrates can be labeled with ^{99m}Tc and Re isotopes via the application of a fac-[^{99m}Tc/Re-(CO)₃]⁺ moiety which coordinates with bidentate and tridentate ligand systems.

Our approach is to attach to glucose a pendent chelating ligand that, in a subsequent reaction, will bind the radioisotope ^{99m}Tc or ^{186/188}Re. Alternatively, a metal-chelate could be preformed and then attached to glucose. To mimic the properties of FDG it is imperative that the effects of the tracer group on the properties of the glucose molecule are minimized. Existing ^{99m}Tc labeled glucose derivatives fail this criterion because they are either ionic or have relatively high molecular weight (i.e., carry two glucose moieties). A versatile low valent fac-{M(CO)₃} core (M=^{99m}Tc¹ or ¹⁸⁶Re¹) was used in these efforts. The facially coordinated carbonyl ligands stabilize the Tc+1 oxidation state, obviating the elaborate, often macrocyclic, polydentate structures required to stabilize other intermediate oxidation states of Tc and Re. In neutral complexes with simple N and O donors the fac-{M(CO)₃} core possesses intermediate lipophilicity, an advantage in living systems.

Glucosamine (2-amino-2-deoxy-D-glucose) is a highly attractive scaffold for a glucosyl ligand, because the amine acts both as a potential coordination site and as a useful target for further functionalization. Furthermore, there is much evidence in the literature to suggest that N-functionalized glucosamines show activity with GLUTs (glucose transporters) and hexokinases—the enzymes that are most closely associated with the metabolism of FDGs even when the functional group is large.

All solvents and chemicals (Fisher, Aldrich) were reagent grade and used without further purification unless otherwise specified. HL¹

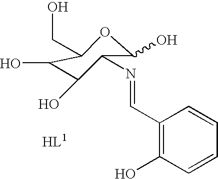

and [NEt₄]₂[Re(CO)₃⁻Br₃] were prepared according to previously published procedures. ¹H and ¹³C NMR spectra were recorded on a Bruker AV-400 instrument at 400.132 and 100.623 MHz, respectively. Assigned chemical shifts for the compounds prepared are recorded below in TABLE 1.

TABLE 1
¹H and ¹³C{¹H} NMR Data (DMSO-d₆) (δ in ppm)
for the α-Anomers of HL² and [(L²)Re(CO)₃]

| | ¹H NMR (δ in ppm) | | | ¹³C{¹H} NMR (δ in ppm) | | |
|---|---|---|---|---|---|---|
| | HL² | [(L²)Re(CO)₃] | δ_{complex} − δ_{ligand} | HL² | [(L²)Re(CO)₃] | δ_{complex} − δ_{ligand} |
| C-1 | 5.11 | 5.22 | 0.11 | 90.4 | 87.5 | −2.9 |
| C-2 | 2.34 | 2.37 | 0.03 | 61.3 | 58.0 | −3.3 |
| C-3 | 3.52 | 3.66 | 0.14 | 72.4 | 79.8 | 7.4 |
| C-4 | 3.06 | 3.20 | 0.14 | 71.0 | 70.6 | −0.4 |
| C-5 | 3.39 | 3.43 | 0.04 | 72.4 | 71.8 | −0.6 |
| C-6 | 3.4, 3.6 | 3.4, 3.6 | | 61.5 | 59.8 | −1.7 |
| C-7 | 3.80 | 3.85, 4.30 | | 48.7 | 51.1 | 2.4 |
| C-8 | | | | 124.8 | 119.4 | −5.4 |
| C-9 | | | | 157.5 | 163.2 | 5.7 |
| C-10 | 6.7 | 6.35 | −0.35 | 119.6 | 120.3 | 0.7 |
| C-11 | 7.05 | 6.80 | −0.25 | 128.9 | 129.1 | 0.2 |
| C-12 | 6.7 | 6.45 | −0.25 | 116.1 | 114.1 | −2.0 |
| C-13 | 7.05 | 6.95 | −0.10 | 129.6 | 130.6 | 1.0 |

Mass spectra (+ ion) were obtained on dilute methanol solutions using a Macromass LCT (electrospray ionization, ESI). Elemental analyses were performed at the University of British Columbia Chemistry Department using Carlo Erba analytical instrumentation. HPLC analyses were performed on Knauer Wellchrom K-1001 HPLC equipped with a K-2501 absorption detector, a Kapintek radiometric well counter, and a Synergi 4 μm C-18 Hydro-RP analytical column with dimensions 250×4.6 mm. The HPLC solvent consisted of 0.1% trifluoroacetic acid in water (solvent A) and acetonitrile (solvent B). Samples were analyzed with a linear gradient method (100% solvent A to 100% solvent B over 30 minutes). The results of this HPLC analysis are reflected below in the Figure.

### Synthesis of N-(2′-Hydroxybenzyl)-2-amino-2-deoxy-D-glucose (HL²)

N-(2′-Hydroxybenzyl)-2-amino-2-deoxy-D-glucose (HL²) was synthesized in the following manner. HL¹ (1.00 g, 3.53 mmol) was dissolved in MeOH (60 mL), and 10% Pd/C w/w (50 mg) was added to the solution to form a reaction mixture. The reaction mixture was stirred under a pressurized H₂ atmosphere (50 bar) for 24 hours and then clarified by filtration and the solvent evaporated to give HL² (0.98 g, 98%) as illustrated below. ESI-MS: 286 ([M+H]+). The calculated analysis for C₁₃H₁₉NO₆.H₂O: C, 51.48; H, 6.98 and N, 4.62. The determined analysis was in close agreement, reflecting: C, 51.50; H, 6.81 and N, 4.60, respectively.

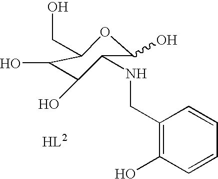

### Synthesis of Tricarbonyl (N-(2′-Hydroxybenzyl)-2-amino-2-deoxy-D-glucose) rhenium(I) (ReL²(CO)₃)

Tricarbonyl (N-(2′-Hydroxybenzyl)-2-amino-2-deoxy-D-glucose) rhenium(I) (ReL²(CO)₃), illustrated below, was prepared by dissolving [NEt₄]₂[Re(CO)₃Br₃] (200 mg, 0.26 mmol), HL² (74 mg, 0.26 mmol) and sodium acetate trihydrate (40 mg, 0.32 mmol) in H₂O (7 mL) and heated with stirring to 50° C. for 2 hours. The solvent was then removed under vacuum and the residue dissolved in CH₂Cl₂ (10 mL) for 30 minutes. On standing, a brown residue was recovered by decanting the solvent. This was purified to an off-white powder (58 mg, 0.10 mmol, 40%) by column chromatography (silica, 5:1 CH₂—Cl₂:CH₃OH). ESI-MS: 556, 554 ([M+H]+), 578, 576 ([M+Na]+). The calculated analysis for C₁₆H₁₈NO₉Re.H₂O: C, 33.57; H, 3.52 and N, 2.45. The determined analysis was in close agreement, reflecting C, 33.55; H, 3.53 and N, 2.75, respectively. 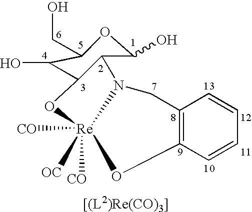
Radiolabeling

[^{99m}Tc(CO)₃(H₂O)₃]⁺ was prepared from a saline solution of Na[^{99m}TcO₄] (1 mL, 100 MBq) using an “Isolink” boranocarbonate kit from Mallinckrodt Inc. Due to the increased chemical inertness and lower redox potential of rhenium, [¹⁸⁶Re(CO)₃(H₂O)₃]⁺ was not accessible by the kit preparation used for technetium. [¹⁸⁶Re(CO)₃(H₂O)₃]⁺ was prepared by addition 4.5 μL of 85% H₃PO₄ to a saline solution of Na[¹⁸⁶ReO₄] (0.5 mL, 100 MBq), followed by addition of this solution to 3 mg of borane ammonia complex that had been flushed with CO for 10 min. The mixture was heated at 60° C. for 15 minutes and then cooled to room temperature. Labeling was achieved by mixing an aliquot of one of the above final solutions (0.5 mL) with a 1 mM solution of HL² in PBS (pH 7.4, 1 mL) and incubating at 75° C. for 30 min.

Stability Evaluation

[(L²)^{99m}Tc(CO)₃(H₂O)] (100 μL, 10 MBq, 1 mM in HL²) was added to 900 μL of either 1 mM histidine or 1 mM cysteine in PBS. The solutions were incubated at 37° C. and aliquots were removed at 1, 4, and 24 hours, at which time HPLC analysis was run. Histidine labeling was achieved by adding a solution containing [^{99m}Tc(CO)₃(H₂O)₃]⁺ to a 1 mM solution of histidine in PBS (pH 7.4, 1 mL) and incubating at 75° C. for 30 minutes. HPLC analysis confirmed the formation of a single radiolabeled product.

The Schiff base formed by condensation of glucosamine with salicylaldehyde HL¹ has been previously investigated as a ligand for transition metals, including ^{99m}Tc(V). Using the starting material [NEt₄]₂[Re(CO)₃ Br₃] as a “cold” surrogate for [M(CO)₃(H₂O)₃]⁺, wherein M is ^{99m}Tc or ¹⁸⁶Re, we synthesized the complex [(L¹)Re(CO)₃] (as observed by ESIMS (+)); however, both the imine and the complex are unstable to hydrolysis and proved to be unsuitable for aqueous radiolabeling chemistry. To circumvent the hydrolysis problem, we reduced HL¹ to the more hydrolytically robust amine phenol HL² (N-(2′-hydroxybenzyl)-2-amino-2-deoxy-D-glucose, Scheme 1). Catalytic hydrogenation of HL¹ provided HL² in 98% yield, with sufficient purity for subsequent radiolabeling studies. The reaction of HL² with [NEt₄]₂[Re(CO)₃Br₃] and NaOAc in H₂O produced the compound [(L²)Re(CO)₃] in 40% yield after column chromatographic purification. The molecular ion was identified as [((L²)Re(CO)₃)+H]⁺ by ESIMS, and the formulation of the bulk sample was confirmed by elemental analysis. Comparison of the anomeric ratio (α/β) observed in the ¹H NMR spectrum (CD₃OD) showed a change from 1.9 for HL² to 1.1 for the complex, indicating that complexation has decreased the difference in thermodynamic stability between the two anomers.

For solubility reasons full NMR studies were carried out in DMSO-d₆ solution (as reflected in TABLE 1). The ¹H NMR spectrum (DMSO-d₆) of the complex is highly convoluted, but the shifting and broadening out of the aromatic resonances compared to those of HL² signify that the phenol “arm” participates, as desired, in the binding of the {Re¹(CO)₃} moiety. The splitting of the methine proton signals into two doublets for each anomer indicates the methine proton inequivalence on formation of the complex. Binding of the ligand N and O donor atoms incorporates the methine in a ring, rigidly holding the two protons in diastereotopic chemical environments. Signals due to the sugar C1 protons were shifted downfield in both anomers compared to those of HL². Peaks due to the sugar C2 protons are also well-resolved and compared to those of HL² are also shifted slightly downfield in both anomers. Small extraneous peaks in the spectrum also indicate that at least one other minor species is present.

When kept overnight in CD₃OD or DMSO-d₆ solution, samples of the complex become visibly brown and the relative intensities of these peaks increase, indicating that they arise from decomposition products. The signals do not correlate with the chemical shifts of uncomplexed HL². Minor species are also detected by UV/visible spectroscopy in the HPLC of the complex and become more significant over time. The ¹³C{¹H} NMR spectrum (d₆-DMSO) of the complex was fully assigned for the α-anomer, and partially assigned for the β-anomer (as reflected above in TABLE 1).

The Re carbonyls show three sharp resonances at 196-198 ppm as expected due to the lack of symmetry. In both anomers, peaks due to the phenol CO and the CH₂ linker are shifted significantly downfield from their values of HL², giving a clear indication that the Re is bound both by the phenol O and glucosamine N.

The C1 and C2 signals of both anomers are shifted upfield on complexation, presumably reflecting some slight conformational change in the hexose skeleton. The result of this could be destabilization of the α-anomer and hence the changed anomeric ratio compared to that of HL² itself. In the α-anomer the C3 signal has shifted downfield 7.4 ppm, suggesting that the C3 glucosamine hydroxyl is binding to the Re center in place of the predicted solvent molecule. Unfortunately, C3 for the β-anomer could not be assigned, due to the lower concentration of the anomer in DMSO solution.

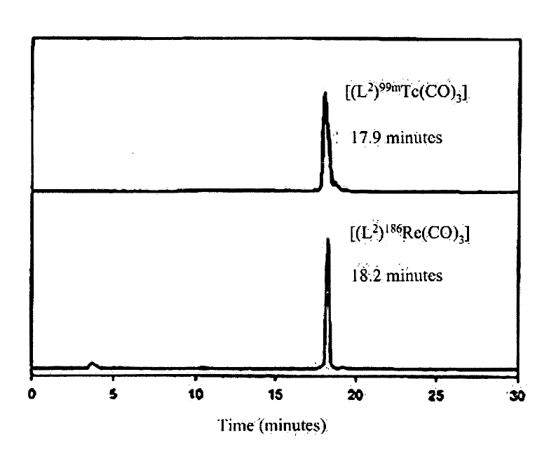

Because it is less polar than either water or methanol, DMSO is generally unable to stabilize the unfavorable dipole moments present in the β-anomer. It is unlikely that the stereochemistry at C1 can have any effect on the geometry-dependent propensity of the C3 hydroxyl to coordinate to Re, thus both anomers are predicted to bind Re in a similar tridentate manner. Labeling HL² with [^{99m}Tc(CO)₃(H₂O)₃]⁺ and [¹⁸⁶Re(CO)₃ (H₂O)₃]⁺ was achieved in 95±2% and 94±3% average radiochemical yields, respectively, as measured by HPLC (an as illustrated in FIG. 1). The identities of the radiolabeled complexes were confirmed to be [(L²)^{99m}Tc(CO)₃] (t_{R}=17.9 minutes) and [(L²)¹⁸⁶Re(CO)₃] (t_{R}=18.2 minutes) by coinjection of the radiolabeled product with the authentic “cold” Re complex (t_{R}=17.9 minutes).

Preliminary assessments of the potential in vivo stability of the ^{99m}Tc complex, cysteine/histidine challenge experiments were then performed. In a typical test, the radiolabeled complex was incubated at 37° C. in aqueous phosphate buffer solution (pH 7.4) containing either 1 mM cysteine or 1 mM histidine, and aliquots were removed at 1, 4, and 24 hours (as reflected in TABLE 2 below). HPLC analysis showed the complex to be stable in either histidine or cysteine solution but only in the short term; by 4 hours, less than 30% of the complex remained intact. Histidine-labeled [^{99m}Tc(CO)₃(H₂O)₃]⁺ was determined to be the major decomposition product of the histidine challenge experiments.

TABLE 2

| | % of [(L²)^{99m}Tc(CO)₃] remaining | | |
|---|---|---|---|
| | 1 hour | 4 hours | 24 hours |
| incubation in cysteine | 88 | 28 | not detected |
| incubation in histidine | 50 | 24 | 4 |

### Percentage of % of [(L²)^{99m}Tc(CO)₃] Remaining After Incubation at 37° C. in 1 mM Cysteine or Histidine for 1, 4 and 24 Hours

The complex instability may be due to the relatively weak binding ability of the donor atoms, especially the secondary amino group and the carbohydrate hydroxyl. When considering modifications to increase complex stability, the fortuitous tridentate binding has directed us to investigate purposely tridentate ligands, and those containing binding groups with higher affinities for the soft {M(CO)₃} center.

In order to address this instability issue, a glucosamine-dipicolylamine conjugate was developed as illustrated below (synthesis VI).

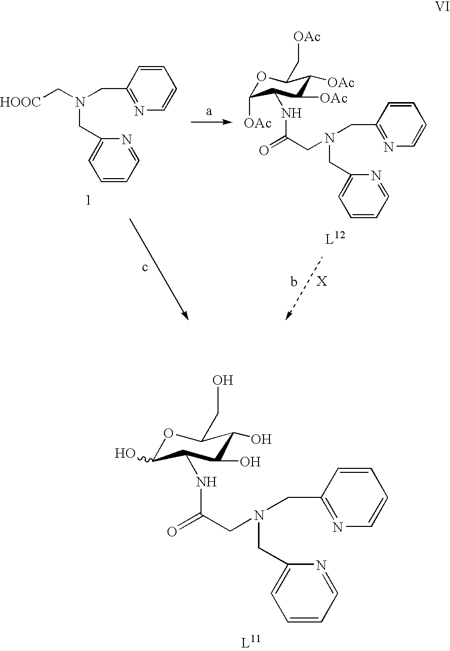

This dipicolylamine derivative formed stable complexes with both ^{99m}Tc and ¹⁸⁶Re as illustrated below.

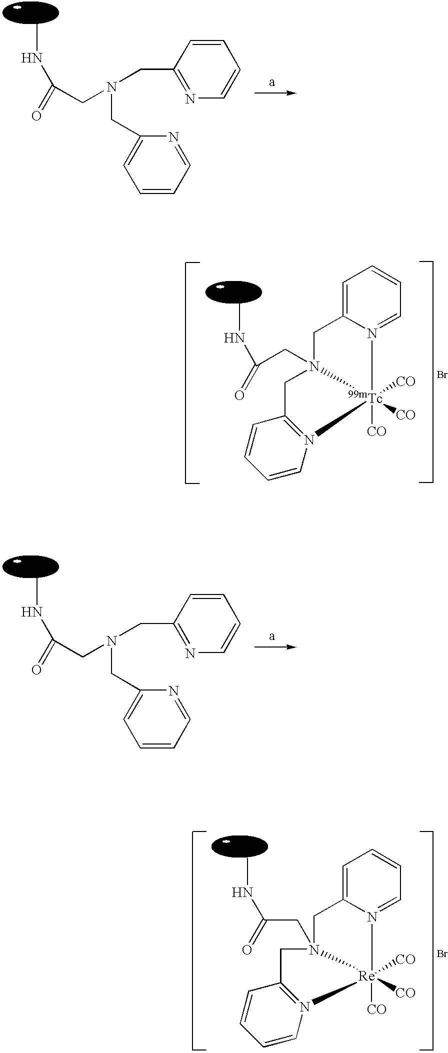

There was virtually no change in these compounds when subjected to cysteine histadine challenge experiments out to 24 hours indicating that these complexes are highly stable. Other tridentate carbohydrate ligands along with different length spacer arms are also being developed as shown in the figures below.
Synthesis of Linkers 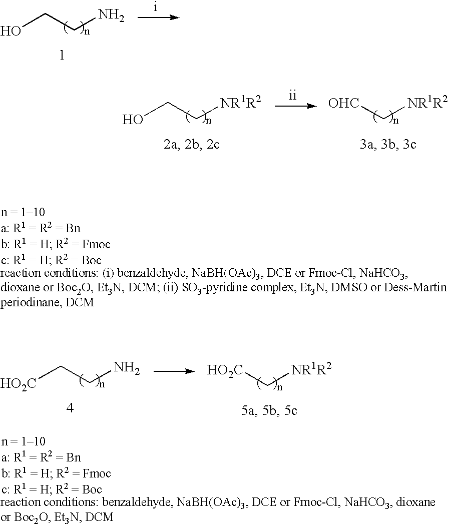
Synthesis of Sugar Precursors

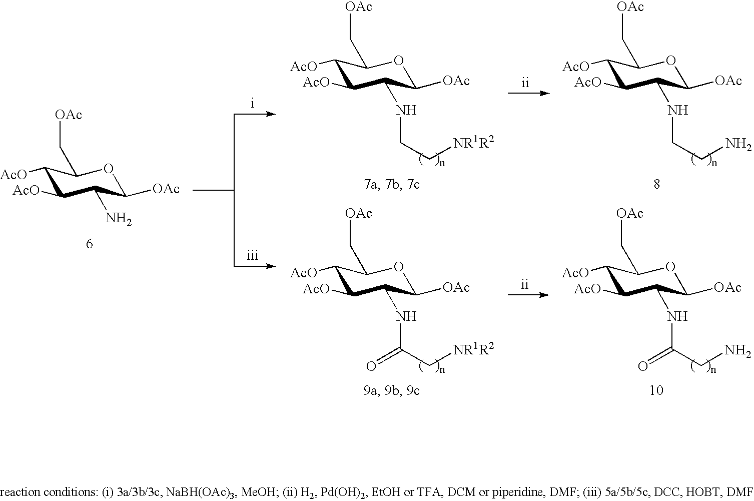

Synthesis of Ligands

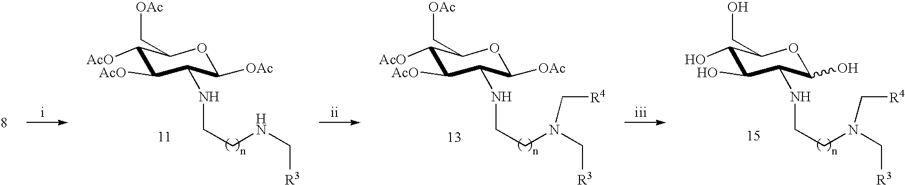

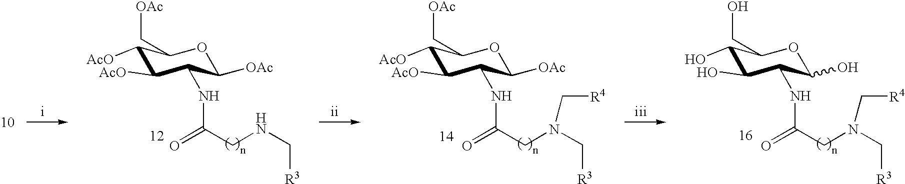

reaction conditions: (i) 2-pyridinecarboxaldehyde/1-benzyl-2-imidazolecarboxaldehye/1-methyl-2-imidazole-carboxaldehyde/imidazolecarboxaldehyde/salicylaldehyde/
17/18/19/20, NaBH(OAc)₃, MeOH; (ii) 2-pyridine-carboxaldehyde/1-benzyl-2-imidazolecarboxaldehye/1-methyl-2-imidazole-carboxaldehyde/
imidazole-2-carboxaldehyde/salicylaldehyde/ 17/18/19/20/3b-1, NaBH(OAc)₃, MeOH or BrCH₂CO₂Et, Na₂CO₃, CH₃CN;
(iii) a. KOH, H₂O; b. piperidine, DMF for 3b-1 derivatives.

| | | | |
|---|---|---|---|
| 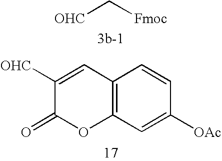 | 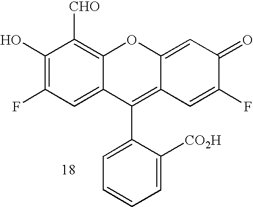 | 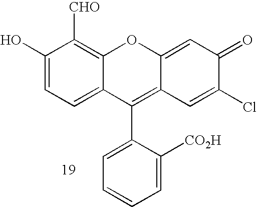 | 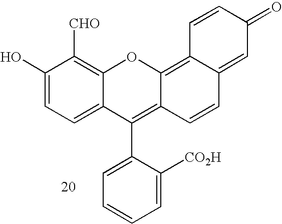 |

| | R³ | R⁴ | | R³ | R⁴ |
|---|---|---|---|---|---|
| 11a/12a | 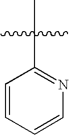 | | | | |
| 11b/12b | 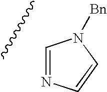 | | | | |
| 11c/12c | 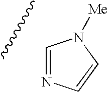 | | | | |
| 11d/12d | 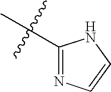 | | | | |
| 11e/12e | 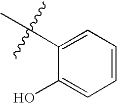 | | | | |
| 11f/12f | 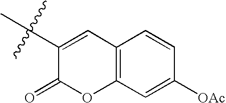 | | | | |
| 11g/12g | 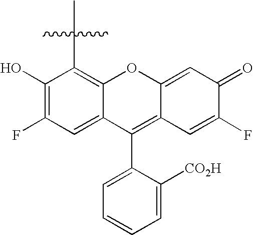 | | | | |
| 11h/12h | 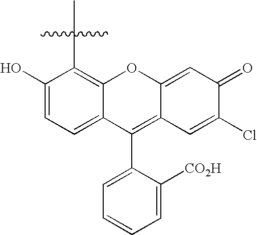 | | | | |
| 11i/12i | 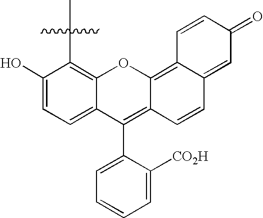 | | | | |
| 13a/14a | 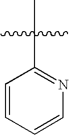 | 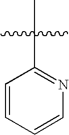 | 15a/16a | 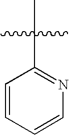 | 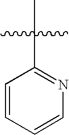 |
| 13b/14b | 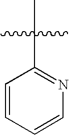 | 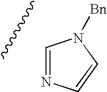 | 15b/16b | 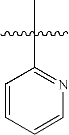 | 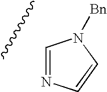 |
| 13c/14c | 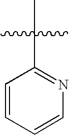 | 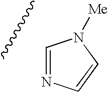 | 15c/16c | 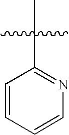 | 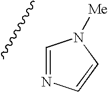 |
| 13d/14d | 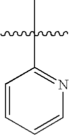 | 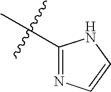 | 15d/16d | 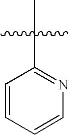 | 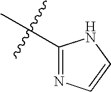 |
| 13e/14e | 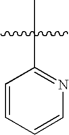 | 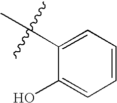 | 15e/16e | 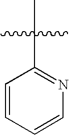 | 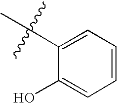 |
| 13f/14f | 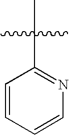 | 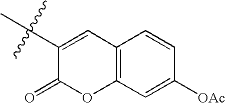 | 15f/16f | 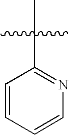 | 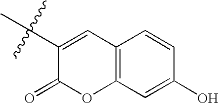 |
| 13g/14g | 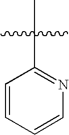 | 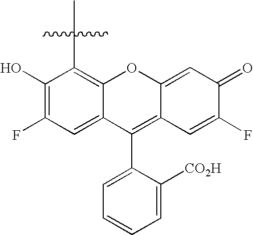 | 15g/16g | 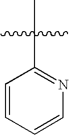 | 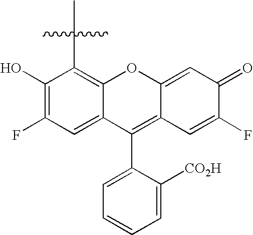 |
| 13h/14h | 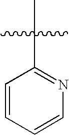 | 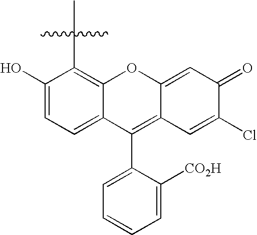 | 15h/16h | 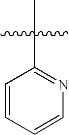 | 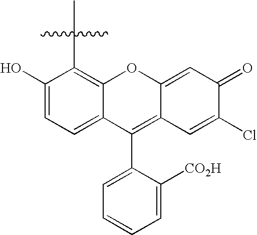 |
| 13i/14i | 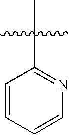 | 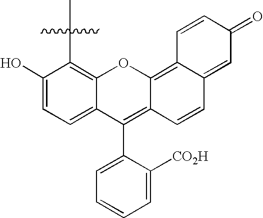 | 15i/16i | 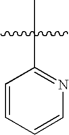 | 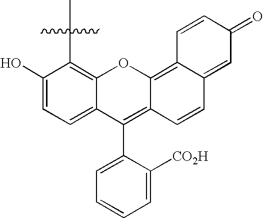 |
| 13j/14j | 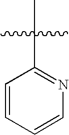 | —CO₂Et | 15j/16j | 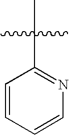 | —CO₂H |
| 13k/14k | 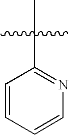 | 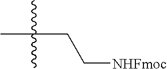 | 15k/16k | 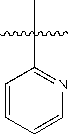 | 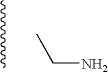 |
| 13l/14l | 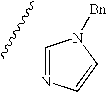 | 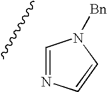 | 15l/16l | 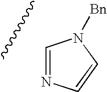 | 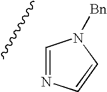 |
| 13m/14m | 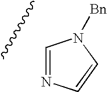 | 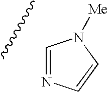 | 15m/16m | 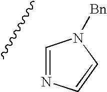 | 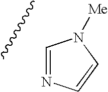 |
| 13n/14n | 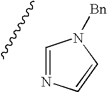 | 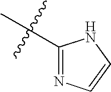 | 15n/16n | 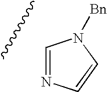 | 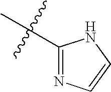 |
| 13o/14o | 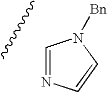 | 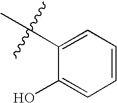 | 15o/16o | 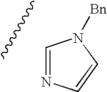 | 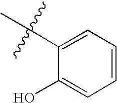 |
| 13p/14p | 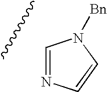 | 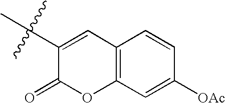 | 15p/16p | 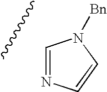 | 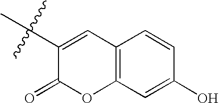 |
| 13q/14q | 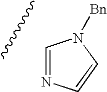 | 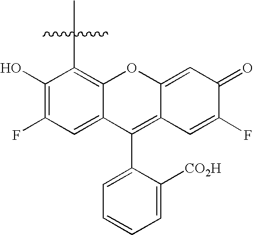 | 15q/16q | 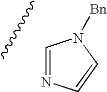 | 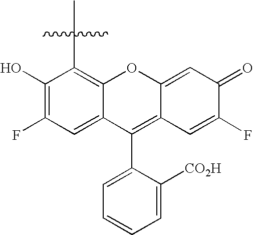 |
| 13r/14r | 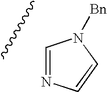 | 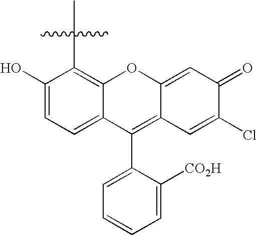 | 15r/16r | 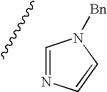 | 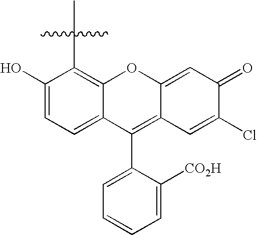 |
| 13s/14s | 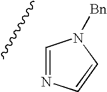 | 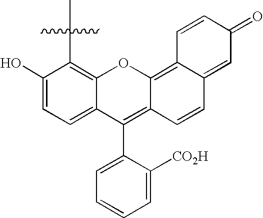 | 15s/16s | 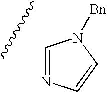 | 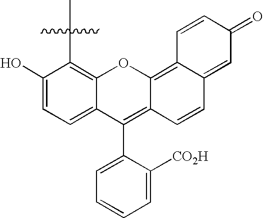 |
| 13t/14t | 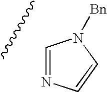 | —CO₂Et | 15t/16t | 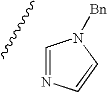 | —CO₂H |
| 13u/14u | 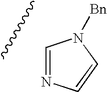 | 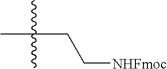 | 15u/16u | 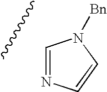 | 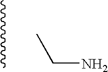 |
| 13v/14v | 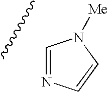 | 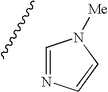 | 15v/16v | 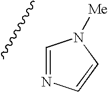 | 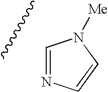 |
| 13w/14w | 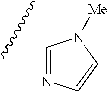 | 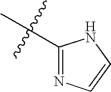 | 15w/16w | 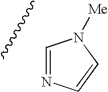 | 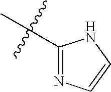 |
| 13x/14x | 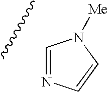 | 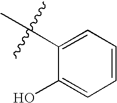 | 15x/16x | 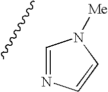 | 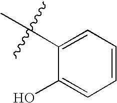 |
| 13y/14y | 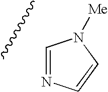 | 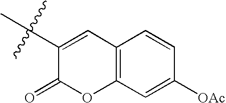 | 15y/16y | 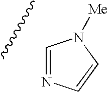 | 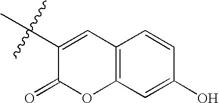 |
| 13z/14z | 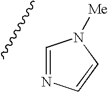 | 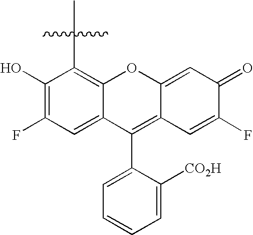 | 15z/16z | 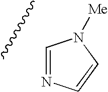 | 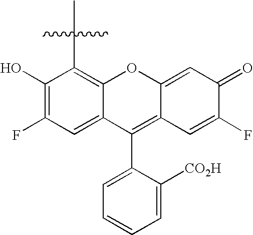 |
| 13aa/14aa | 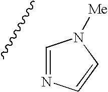 | 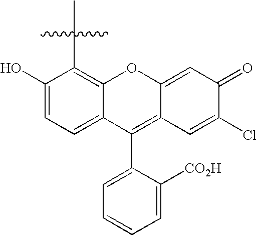 | 15aa/16aa | 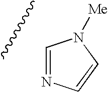 | 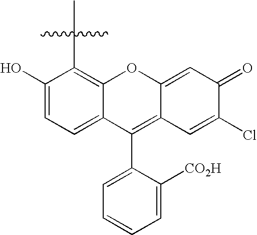 |
| 13ab/14ab | 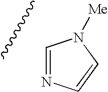 | 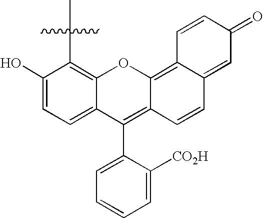 | 15ab/16ab | 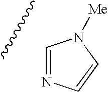 | 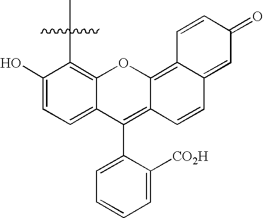 |
| 13ac/14ac | 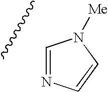 | —CO₂Et | 15ac/16ac | 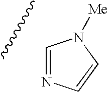 | —CO₂H |
| 13ad/14ad | 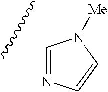 | 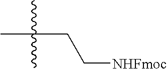 | 15ad/16ad | 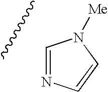 | 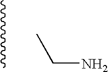 |
| 1ae/14ae | 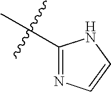 | 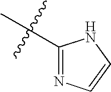 | 15ae/16ae | 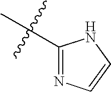 | 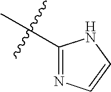 |
| 13af/14af | 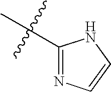 | 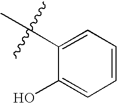 | 15af/16af | 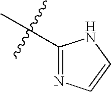 | 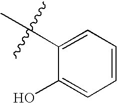 |
| 13ag/14ag | 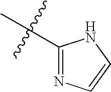 | 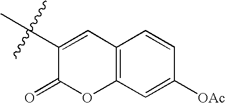 | 15ag/16ag | 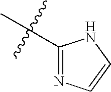 | 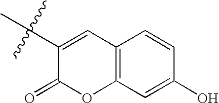 |
| 13ah/14ah | 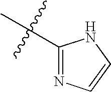 | 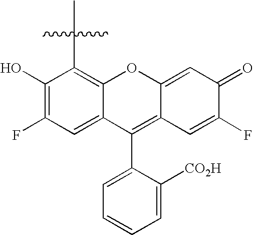 | 15ah/16ah | 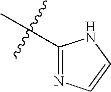 | 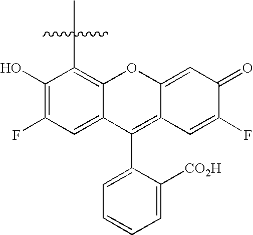 |
| 13ai/14ai | 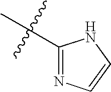 | 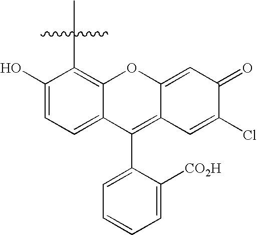 | 15ai/16ai | 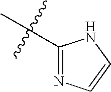 | 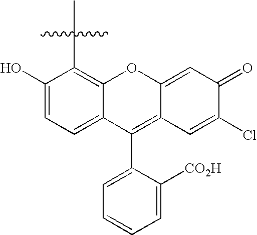 |
| 13aj/14aj | 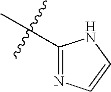 | 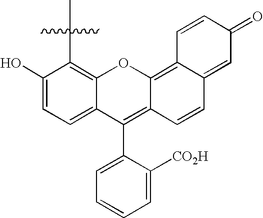 | 15aj/15aj | 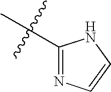 | 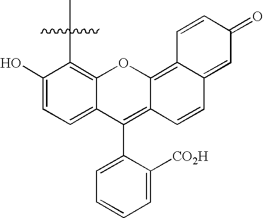 |
| 13ak/14ak | 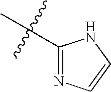 | —CO₂Et | 15ak/16ak | 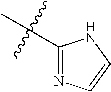 | —CO₂H |
| 13al/14al | 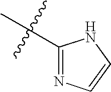 | 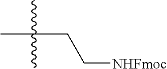 | 15al/16al | 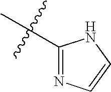 | 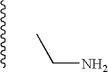 |
| 13am/14am | 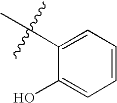 | 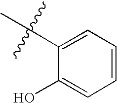 | 15am/16am | 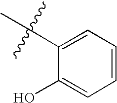 | 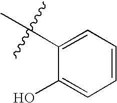 |
| 13an/14an | 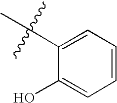 | 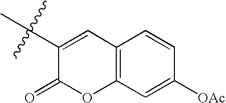 | 15an/16an | 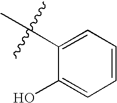 | 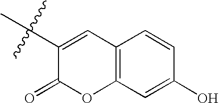 |
| 13ao/14ao | 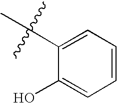 | 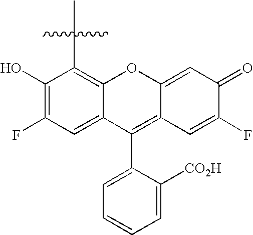 | 15ao/16ao | 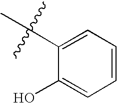 | 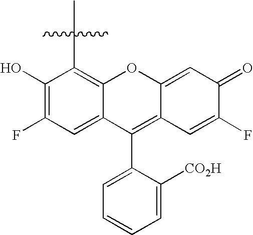 |
| 13ap/14ap | 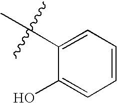 | 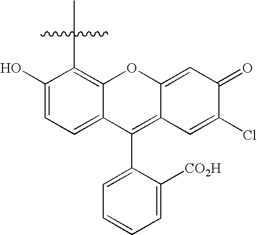 | 15ap/15ap | 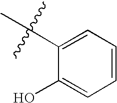 | 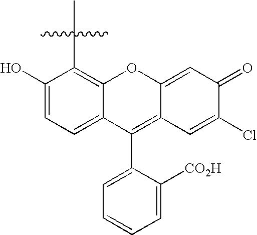 |
| 13aq/14aq | 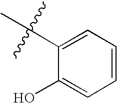 | 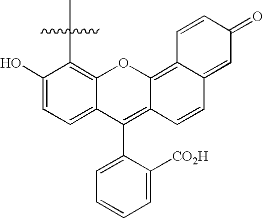 | 15aq/16aq | 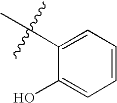 | 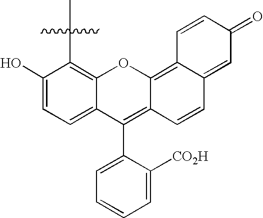 |
| 13ar/14ar | 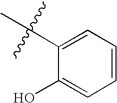 | —CO₂Et | 15ar/16ar | 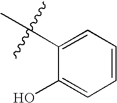 | —CO₂H |
| 13as/14as | 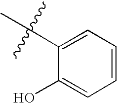 | 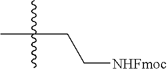 | 15as/16as | 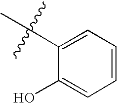 | 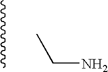 |
| 13at/14at | 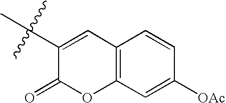 | —CO₂Et | 15at/16at | 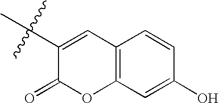 | —CO₂H |
| 13au/14au | 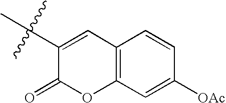 | 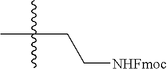 | 15au/16au | 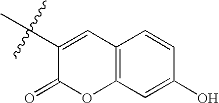 | 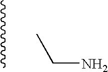 |
| 13av/14av | 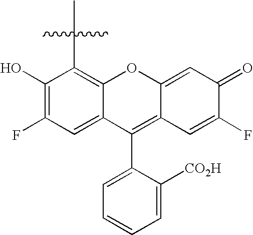 | —CO₂Et | 15av/16av | 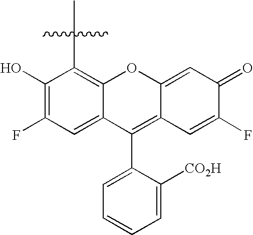 | —CO₂H |
| 13aw/14aw | 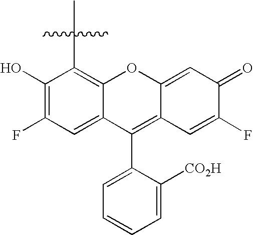 | 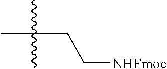 | 15aw/16aw | 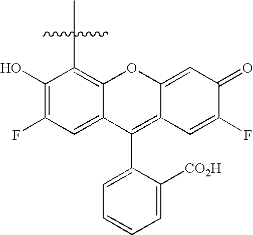 | 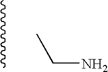 |
| 13ax/14ax | 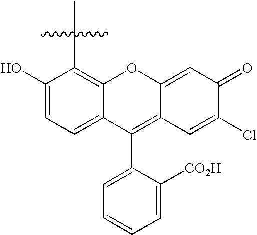 | —CO₂Et | 15ax/16ax | 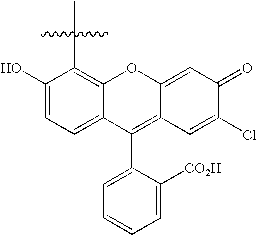 | —CO₂H |
| 13ay/14ay | 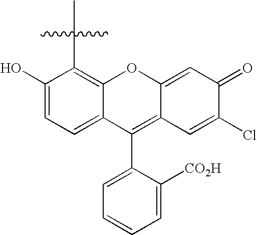 | 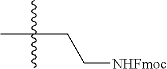 | 15ay/16ay | 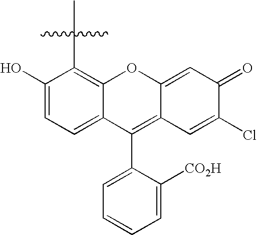 | 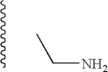 |
| 13az/14az | 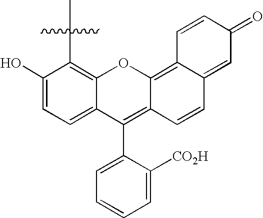 | —CO₂Et | 15az/16az | 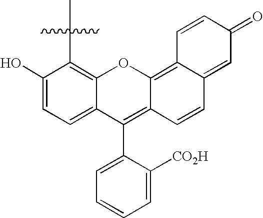 | —CO₂H |
| 13ba/14ba | 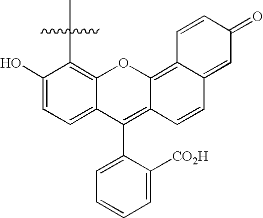 | 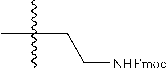 | 15ba/16ba | 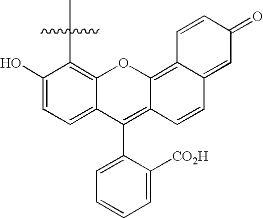 | 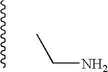 |

Materials. All solvents and reagents were used as received. 1 wherein n 1-5, 7 and 8; 2b with n=1, 2 and 5; 2c with n=1-5; 4 with n=0-7, 9 and 10; 5b/5c with n=2-7 and 10 are commercially available (Acros, Aldrich, TCI, Fluka). Compound types 2a, 2b, 2c, 3a, 3b, 3c were prepared as described in White, J. D.; Hansen, J. D., J. Org. Chem. 2005, 70, 1963-1977 and 5a as described by Breitenmoser, R. A.; Heimgartner, H., Helv. Chim. Acta 2001, 84, 786-796, the contents of which are incorporated herein, in their entirety, by reference. Various of the known compounds 6 (Silva, 1999), 17 (Lim, 2005), 18 and 20 Chang, C. J. et al., Inorg. Chem. (2004), 43, 6774-6779, and Chang, C. J. and Jaworski, J. et al., Proc. Natl. Acad. Sci. (2004) 101, 1129-1134 and 19 Nolan, E. et al., J. Inorg. Chem. (2004), 43, 2624-2635 were prepared as described in the corresponding reference. Those skilled in the art may, of course, develop additional synthesis and/or preparation techniques for producing these and related compounds.
Experimental
General Procedure for Preparation of 2a.

To ethanolamine in 1,2-dichloroethane, benzaldehyde is added and allowed to stir at ambient temperature under N₂. Sodium triacetoxyborohydride is then added and the reaction is further stirred for a period of time. The reaction is quenched by addition of aqueous Na₂CO₃ and then partitioned, the aqueous phase subsequently extracted with CH₂Cl₂. The combined organic extracts is washed with brine and dried with MgSO₄. The resulting solution is taken to dryness by rotary evaporation and 2a is isolated using column chromatography.

General Procedure for Preparation of 2b.

To a solution of 1,4-dioxane containing ethanolamine and NaHCO₃, is added Fmoc-Cl and allowed to stir at ambient temperature under N₂. The reaction is stirred for a period of time, the resulting solid filtered and the filtrate reduced to dryness by rotary evaporation. 2b is isolated using column chromatography.

General Procedure for Preparation of 2c.

To a solution of CH₂Cl₂ containing ethanolamine and Et₃N, is added Boc₂O and allowed to stir at ambient temperature under N₂. The reaction is stirred for a period of time and taken to dryness by rotary evaporation. The resulting oil is taken up in CH₂Cl₂ and washed with aqueous Na₂CO₃, brine and dried with MgSO₄. The solvent is taken off under reduced pressure and 2c is isolated using column chromatography.

General Procedure for Preparation of 7.

To freebased 1,3,4,6-tetra-O-acetyl-2-deoxy-glucosamine 6 (prepared by dissolving 6.HCl in aqueous Na₂CO₃ and extracting into CH₂Cl₂, then evaporated to dryness) is added freshly prepared 3a. The resulting solution is stirred at ambient temperature under N₂ followed by the addition of NaBH(OAc)₃. The reaction is quenched by addition of aqueous Na₂CO₃ and the resulting mixture partitioned. The aqueous phase is further extracted with CH₂Cl₂. The combined organic extracts is washed with brine and dried with MgSO₄. Rotary evaporation followed by column chromatography afforded pure 7a.

General Procedure for Preparation of 9.

To a cold solution of 5a in CH₂Cl₂ under Ar is added DCC followed by HOBT in DMF. After keeping the low temperature for a period of time, freebased 1,3,4,6-tetra-O-acetyl-2-deoxy-glucosamine 6 is added. The reaction is then allowed to warm to room temperature and stirred for an additional amount of time. The solid by-products are filtered off, the filtrate concentrated under reduced pressure and 9a is isolated by column chromatography.

General Procedure for Preparation of 8/10 from 7a/9a.

To a solution of 7a in MeOH is added Pd(OH)₂. Reduction with H₂ is done at 1 atm. The reaction mixture is filtered through a pad of celite previously washed with methanol and rotary evaporation of the solvent afforded 8.

General Procedure for Preparation of 8/10 from 7b/9b.

7b is dissolved in CH₂Cl₂ and TFA is added. The resulting solution is stirred at ambient temperature under N₂ for a period of time. The solution is taken to dryness by rotary evaporation and the resulting residue is taken up in CH₂Cl₂, washed with aqueous NaHCO₃, brine and dried with MgSO₄. Evaporation of the solvent followed by column chromatography afforded pure 8.

General Procedure for Preparation of 8/10 from 7c/9c.

7c is dissolved in DMF and piperidine is added. The resulting solution is stirred at ambient temperature under N₂ for a short period of time and is taken to dryness by rotary evaporation. Pure 8 was isolated by column chromatography.

General Procedure for Preparation of 11/12 from 8/10.

To a solution of 8a in 1,2-dichloroethane is added 2-pyridinecaboxaldehyde. The resulting solution is stirred at ambient temperature under N₂ for a short period of time followed by the addition of NaBH(OAc)₃. The reaction is quenched by the addition of aqueous Na₂CO₃. The aqueous phase is extracted with CH₂Cl₂ and the combined extracts is washed with brine and dried with MgSO₄. Rotary evaporation of the solvent afforded crude 11a which is isolated by column chromatography.

General Procedure for Preparation of 13/14 from 11/12.

To a solution of 11a in 1,2-dichloroethane is added salicylaldehyde. The resulting solution is stirred at ambient temperature under N₂ for a short period of time followed by the addition of NaBH(OAc)₃. The reaction is quenched by the addition of aqueous Na₂CO₃. The aqueous phase is extracted with CH₂Cl₂ and the combined extracts is washed with brine and dried with MgSO₄. Rotary evaporation of the solvent afforded crude 13e which is isolated by column chromatography.

General Procedure for Preparation of 15/16 from 13/14.

To a solution of 13e in MeOH is added 1M KOH. The resulting solution is stirred at ambient temperature for a period of time. The reaction mixture is neutralized with 1M HCl and taken to dryness under reduced pressure. The resulting residue is taken up in water and passed through REXYN(H). Evaporation of the solvent afforded 15e.

In summary, neutral, low molecular weight ^{99m}Tc-labeled and ¹⁸⁶Re-labeled carbohydrate complexes were produced in high radiochemical yield from a simple functionalized glucosamine. HL² is in trials as a ligand for ^{62/64}Cu and ⁶⁷¹⁶⁸Ga, and other carbohydrate-containing ligands for ^{99m}Tc and ^{186/188}Re are under study.

A number of references are identified in the provisional application from which this application claims priority. Although the present disclosure, in light of the knowledge regarding synthesis, isolation and characterization procedures attributed to those skilled in the art of synthesizing such compounds, is believed sufficient to allow those skilled in the art to practice the invention, each of those references is incorporated, in its entirety, by reference. To the extent that the level of ordinary skill is not as advanced as believed, any material disclosed in the listed references that may subsequently be deemed essential to practicing the invention, such material will be incorporated into the present application without constituting the introduction of new material.

We claim:

1. A method for synthesizing a radiolabeled sugar-metal complex comprising:
synthesizing a sugar precursor;
synthesizing a chelating ligand;
reacting the sugar precursor and the chelating ligand to form a sugar-metal complex; and
labeling the sugar-metal complex with a radioisotope to obtain the radiolabeled sugar-metal complex.

2. The method for synthesizing radiolabeled sugar-metal complexes according to claim 1, wherein:
the radioisotope is selected from a group consisting of the ^{99m}Tc or Re isotopes

3. The method for synthesizing radiolabeled sugar-metal complexes according to claim 1, wherein:
the sugar-metal complex includes a bidentate or tridentate ligand system.

4. The method for synthesizing radiolabeled sugar-metal complexes according to claim 1, wherein:
the chelating ligand includes iron (Fe).

5. The method for synthesizing radiolabeled sugar-metal complexes according to claim 1, wherein:
the chelating ligand is a ferrocene.

6. The method for synthesizing radiolabeled sugar-metal complexes according to claim 1, wherein:
the radiolabeled sugar-metal complex is soluble in water.

7. The method for synthesizing radiolabeled sugar-metal complexes according to claim 1, wherein:
the radiolabeled sugar-metal complex is insoluble in water.
